(19) United States
(12) Patent Application Publication
(10) Pub. No.: US 2021/0033620 A1
(43) Pub. Date: Feb. 4, 2021

(54) Immobilized Enzymatic Digestion of Blood Products for Diagnostic Testing

(71) Applicants: Marc David Porter, Park City, UT (US); Lars Bjorn Laurentius, Cottonwood Heights, UT (US); Nicholas Owens, Sacramento, CA (US); Ryan Evan Robinson, Taylorsville, UT (US)

(72) Inventors: Marc David Porter, Park City, UT (US); Lars Bjorn Laurentius, Cottonwood Heights, UT (US); Nicholas Owens, Sacramento, CA (US); Ryan Evan Robinson, Taylorsville, UT (US)

(73) Assignee: University of Utah, Salt Lake City, UT (US)

(21) Appl. No.: 16/942,724

(22) Filed: Jul. 29, 2020

Related U.S. Application Data
(60) Provisional application No. 62/879,814, filed on Jul. 29, 2019.

(51) Int. Cl. G01N 33/68; G01N 33/58

(52) U.S. Cl. CPC G01N 33/6842; G01N 2333/96433; G01N 33/581

(57) ABSTRACT

This invention discloses a pretreatment approach for blood and bodily fluids to remove unwanted protein interferences in the measurement of analytes. Enzymes either contained in a cartridge or immobilized on a solid support break down proteins that complex with the analyte to shield it from detection. This pretreatment significantly enhances the detectability of analytes and does not require subsequent clean-up steps that would normally be required to ensure the functionality of the analysis method, thereby, creating a simple yet powerful approach for sample pretreatment in a variety of settings ranging from a complex laboratory infrastructure to a field deployable application.

### REFERENCE TO RELATED APPLICATION

This application claims inventions disclosed in Provisional Patent Application No. 62/879,814, filed Jul. 29, 2019, entitled IMMOBILIZED ENZYMATIC DIGESTION OF BLOOD PRODUCTS FOR DIAGNOSTIC TESTING.” The benefit under 35 USC § 119(e) of the above-mentioned United States Provisional Applications is hereby claimed, and the aforementioned applications are hereby incorporated herein by reference.

### FIELD OF INVENTION

This invention relates to the pretreatment of blood and other body fluid samples as a means to remove components that interfere in the diagnostic testing for markers of diseases and cancer, and other maladies.

### BACKGROUND

The measurement of biomarkers in body fluid samples can be challenging as the complexity of these sample matrices can interfere with selective analyte detection. This applies to both laboratory-based and field-deployable tests. The impact of these interferences can be overcome either by diluting the sample in a more innocuous solution or by the addition of reagents that mask, block, or otherwise disrupt the mechanistic process causing the interference. These approaches, however, result in an overall loss of measurement sensitivity. By employing a simple enzymatic pretreatment step that is confined to a solid support, this issue can be overcome, thereby improving the analytical sensitivity of the measurement and its limit of detection (LOD). The capabilities of this approach, which is applicable to a wide range of analyses in the analytical, bioanalytical, and combinatorial sciences, is demonstrated by way of example for the detection of mannose-capped lipoarabinomannan (LAM), a marker of tuberculosis (TB) infection, when spiked into human serum and measured by an enzyme-linked immunosorbent assay (ELISA).

### SUMMARY OF THE INVENTION

The challenges faced in the detection of infectious disease, cancer, and other markers of human and animal maladies can be compromised by the complexation of the marker when attempting to detect its presence by an immunoassay or other type of selective recognition pathway from complex sample matrices (e.g., whole blood, plasma, serum, urine or other specimens). This invention discloses an approach to break up such complexes by an enzymatic digestion step that flows the sample through high capacity solid phase materials modified with a layer of protease enzymes.

### BRIEF DESCRIPTION OF THE FIGURES

The accompanying figures, when linked with the detailed descriptions that follow, serve to illustrate various embodiments of the invention, which aid in framing the operational principles and associated advantages of the invention.

FIG. 1 illustrates a process in which an immobilized enzyme digests immunocomplexers by peptide cleavage that interferes with the detection of a complexed marker. The design can include: (1) a membrane positioned either upstream or downstream of the digestion membrane that filters the sample to remove dissolved solids and other forms of sample debris, and/or (2) an upstream or downstream membrane modified with a readily dissolvable reagent in a dried (e.g., powder) form that upon dissolution in the liquid sample, alters the chemical properties of the sample (e.g., pH or ionic strength) as necessary to facilitate the analysis of the sample by ELISA and other forms of a diagnostic test;

FIG. 2 presents % recovery data from ELISA measurements on LAM spiked into pooled human (healthy patient) serum after pretreatment with perchloric acid (PCA), and proteinase K (PK). The recoveries are calculated by comparison to the ELISA responses for LAM spiked into PBS (10 mM, pH 7.4) with 1% BSA;

FIG. 3 presents ELISA responses for LAM spiked into pooled human (healthy patients) serum, followed by a heterogeneous digestion using Eupergit®C particle-based PK digestions or by a homogeneous reaction with PK dissolved directly in the sample solution.

FIG. 4 illustrates an example of a pretreatment approach in which the enzymatic component of the digestion architecture is immobilized on a solid phase extraction membrane (SPME), which acts to break down proteins into small peptide fragments as the sample passes through the membrane, thereby releasing the analyte from its protein-based complex and facilitating detection by an immunoassay and other types of diagnostic tests.

### DETAILED DESCRIPTION

By way of context, the embodiments of the present invention are described within the framework of a heterogeneous immunoassay. It should, however, be readily recognized by practitioners skilled in the art that these embodiments apply well beyond this illustrative example to include the use of this invention across all areas of investigative and applied measurement science and technology.

Note that relational terms such as “first” and “second,” “top” and “bottom”, and the like may be used solely to distinguish one entity or action from another entity or action without necessarily requiring or implying an actual relationship or order between such entities or actions. The terms “comprises,” “comprising,” or any variations thereof, are intended to cover a non-exclusive inclusion such that a process, method, article, or apparatus that consists of a number of different and/or related elements is not limited to only those elements but may include other elements not expressly listed or inherent to such a process, method, article, or apparatus. An element preceded by “comprises” does not, without more constraints, preclude the existence of a number of additional identical elements in the process, method, article, or apparatus that comprises the element.

Breakthroughs in tuberculosis (TB) diagnostics remain a major global health priority. As a diagnostic marker for TB, mannose-capped lipoarabinomannan (LAM), is a highly branched lipoglycan (17±5 kDa) that is unique to mycobacteria and is a major virulence factor in the infectious pathology of TB. LAM is: (1) a significant (˜40%), but loosely associated, component of the mycobacterial cell wall; (2) easily shed into the circulatory system; (3) present in the serum and urine of TB-infected patients; and (4) considered an important and much needed marker for active TB infection. Work has shown, however, that the capture and/or labeling steps in a sandwich immunoassay for LAM, when using serum and urine from TB-infected patients, are sterically hindered by its immunocomplexation. This invention disclosure describes a method that overcomes the immunocomplexation challenge in a manner that does not alter the binding affinity of LAM in the capture and/or binding steps in an immunoassay, which, as will be shown, facilitates the detection of LAM.

The purification and extraction of TB antigens for the purposes of diagnostic testing is a developing field. Recent work has focused on using the acidification of serum and urine as a means to induce protein denaturation, which releases LAM from immunocomplexation. This approach to sample pretreatment, while notably improving the detection of LAM, recovers only ˜20% recovery of LAM when spiked into serum samples when compared to LAM spiked into phosphate buffered saline (PBS, 10 mM, pH 7.4). These low recoveries are, in large part due to the hydrolytic degradation of LAM in acidic solutions. Work has also shown that heating serum samples, which induces protein denaturation, can improve detectability, but not to the same labels as acidification.

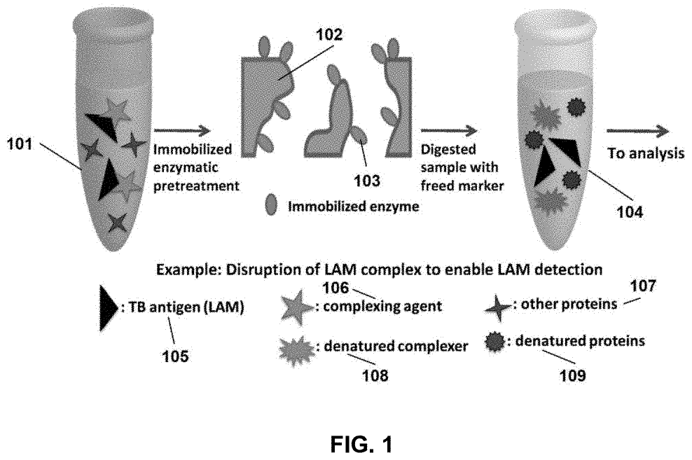

As an alternative to the above pretreatment approaches, FIG. 1 shows how protease enzymes like proteinase K (PK) can be used to break up any immunocomplexed LAM by peptide cleavage. Importantly, PK and other enzymes selectively cleave only peptide linkages. LAM, being a glycolipid, is therefore not susceptible to the enzymatic action of proteases. This approach to freeing LAM from the steric hindrance presence by immuncomplexation will therefore result in increased levels of LAM recovery, and, as a result, improvements in LAM detectability.

For context, PK is an example of an enzyme that is useful for general digestion of proteins in biological and other media. It is a serine protease that hydrolyzes a wide range of peptide linkages. PK is active over a wide range of temperatures and values of solution pH, with an optimal activity between 20 and 55° C., and pH values between 7.5 and 12. The enzymatic activity of PK can be enhanced by additives like sodium dodecyl sulfate (SDS), urea, and dithiothreitol (DTT). Calcium stabilizes PK, but does not alter its catalytic activity. PK, when frozen in aqueous solution at −20° C., remains stable for at least 2 years. It is commonly used to digest residual amounts of protein when preparing patient samples for nucleic acid analysis, but has not been applied to pretreating samples with high protein content of whole blood, human plasma, and human serum.

In the pretreatment protocol illustrated in FIG. 1, the sample solution (101) is exposed to a solid support modified with PK (102) to free LAM (105) by cleaving complexing agents (106) and other proteins (107). This results in a pretreated sample solution (104) containing denatured complexes (108), other denatured proteins (109), and free LAM (105). For perspective, this document discusses two approaches carrying out a PK digestion: a homogeneous process in which the sample is digested by dissolving PK directly in the sample and the heterogeneous process shown in FIG. 1. The results from the homogeneous reaction process are used as a comparator to the findings from the heterogeneous reaction process. Note that the protocol uses PK immobilized on a solid support, which eliminates the need to apply any subsequent processing steps needed to deactivate any PK that may attack the antibodies used in the LAM capture and labeling steps, which would degrade the performance of the immunoassay.

To identify the most effective conditions for the homogeneous digestion of undiluted human serum spiked with LAM, the impact of temperature, PK concentration, digestion time, and PK inactivation steps were investigated. In nucleic acid purification protocols, PK concentrations typically range between 50 and 200 μg/mL. For undiluted human serum, PK concentrations ranging from 20-400 μg/mL worked to varying degrees, with a concentration of 200 μg/mL yielding the highest recovery of LAM. PK was also found to digest proteins at room temperature, but that elevations in temperature increased the rate of LAM digestion, which was assessed by determinations of the recovery of LAM by ELISA. By way of reference, a 10° C. rise in temperature increases the activity of most enzymes by 50 to 100%. The most effective temperature for digesting undiluted human serum was found to be 50° C. Studies also showed that the most effective incubation time was 30 min, with longer times resulting in aggregated protein fragments that interfered with the immunoassay. Collectively, the optimal conditions for carrying out the digestion of human serum spiked with LAM included a 200 μg/mL concentration of PK at an incubation time of 30 min and a temperature of 50° C. This is followed by a heat inactivation step for the PK at high temperature (95-100° C.) for 10 min. The volume of liquid recovered after sample centrifugation from a 1.0 mL serum sample typically ranged from 0.75 to 0.80 mL.

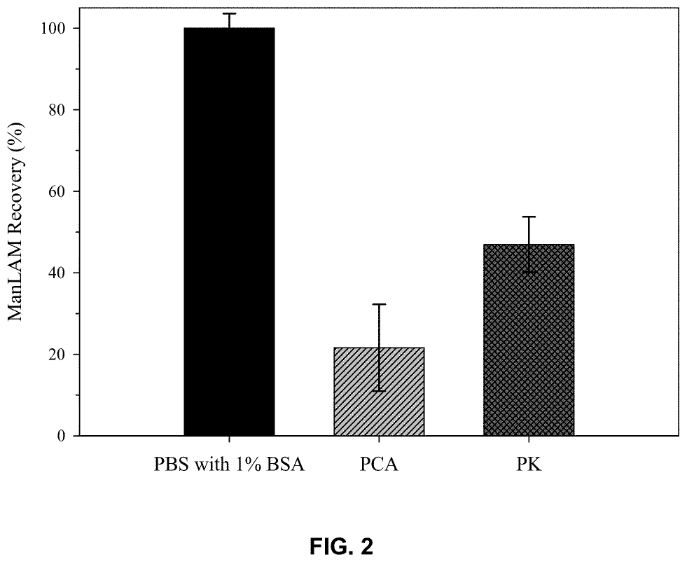

FIG. 2 shows the recovery of LAM for two different pretreatment approaches in human serum samples. In order to validate the pretreatment effectiveness, LAM was dissolved in a solution that yielded the highest signal, free of immunocomplexers while providing a pH, ionic strength, and protein content similar to that of body fluids. This solution was selected to be PBS buffer (10 mM) with 1% BSA at a pH of 7.4. Consequently, pretreatment approaches are evaluated based on comparing the response of LAM at a specific concentration in human serum after pretreatment to LAM in buffer, which can be used to calculate the percentage of LAM recovery. For reference, recoveries for LAM spiked into pooled human serum and pretreated by acidification with perchloric acid (PCA) yielded recoveries of ˜20% which highlights the susceptibility of LAM to hydrolytic degradation at low pH. In comparison, the enzymatic pretreatment with PK gave a recovery of ˜50%, with an improvement in the limit of detection (LOD) by ˜25 times when compared to that with acidification pretreatment.

While there still appears to be room to improve the recovery of LAM, which could be achieved, for example by incorporating SDS or other additives that increase the activity of PK, it is also possible that the ELISA measurements used to assess recovery of LAM were compromised by the presence of small amounts of PK that were not fully deactivated by the heat-based deactivation step. Any residual PK could then enzymatically degrade the tertiary structure of the immobilized antibodies, which would negatively bias the amount of measured LAM.

To address this issue, an approach was developed that used PK immobilized on a solid support, which inherently eliminates the possible impact of any residual, active PK on the downstream measurements by ELIA. This approach may also prove more effective by enabling a higher level of enzyme loading than possible for the analogous homogeneous process, which is limited by enzyme solubility. Taken together, this approach will result in faster and more efficient digestion, while also eliminating the need for an enzyme deactivation step post digestion, and as often found for immobilized enzyme products, a prolonged enzyme shelf-life.

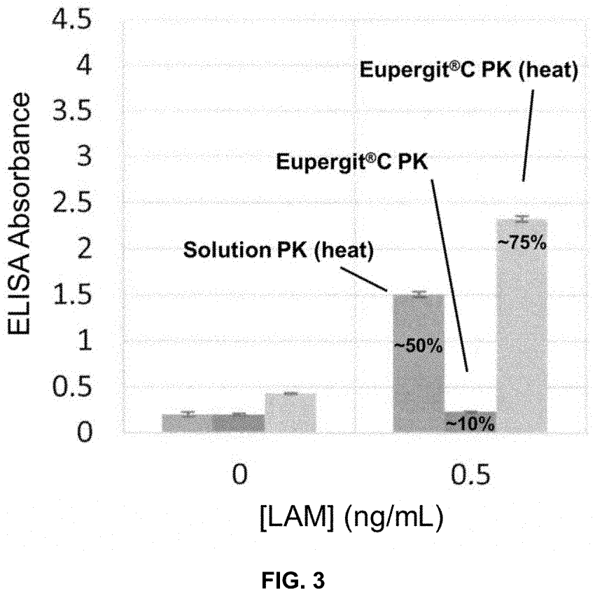

The principle of this approach is demonstrated in FIG. 3 by using macroporous particles called Eupergit®C comprised of immobilized PK. The effectiveness of using the particles in sample pretreatment was compared to solution-based PK pretreatment in the detection of LAM spiked into pooled human serum at a concentration of 0.5 ng/mL. The ELISA response was used to calculate the percentage of LAM recovery, which is the same approach used for the data analysis for FIG. 2. The solution-based PK approach requires heat for inactivation and yields a recovery of ˜50%. When using PK-immobilized Eupergit®C particles without a subsequent heating step, the recovery decreases to ˜10%; this is the result of the efficient protein digestion by the immobilized PK, which creates a sample solution so rich in small fragments that the accumulation of these fragments on the capture surface of the wells in the microplate used in the ELISA measurements severely compromises the analysis. However, when using PK-immobilized Eupergit®C particles with a subsequent heating step, the recovery significantly increases to ˜75%. The heating step removes the small, agglomerated peptide fragments that interfere with the immunoassay. This demonstrates the effectiveness of a solid support-immobilized enzymatic digestion for sample pretreatment.

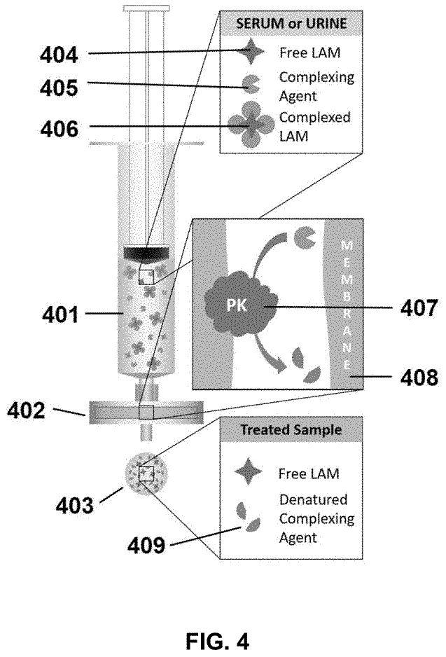

The application of immobilized enzymes in sample pretreatment can easily be applied to laboratory-based tests, and will also be of real value to point-of-care (POC) or field-deployable tests for TB and a number of other markers (e.g., galactomannan, a marker for invasive aspergillus infections) that are difficult to quantify due to immunocomplexation. It should also be noted that the use of immobilized enzymes reduces the number of sample handling/manipulation steps. In these situations, a simple cartridge that can either be free-standing or incorporated into an assay would be ideal. The concept is illustrated in FIG. 4, wherein a sample contained in a syringe (401) is passed through a syringe filter (402) that contains an inert, solid support membrane (408) to which enzymes (407) are immobilized. The sample contains free analyte (404), complexing agents (405), and complexed analyte (406). When the sample flows through the pretreatment device, the enzymes immobilized on the solid support digest proteinaceous complexing agents by peptide cleavage, resulting in free analyte (404) and denatured complexing agents (409). In doing so, the simple pretreatment approach can disrupt complexing agents, enabling the low-level detection of critical biomarkers at a POC setting. Such an approach can also be easily integrated into a microfluidic-based test offering an all-in-one solution. Note also that this process extends well beyond that for the pretreatment of samples for the detection of LAM, which is an important marker of TB infections. It also has utility to the facilitation of a number of other carbohydrate and glycolipid markers, including those for E. coli, leprosy, Streptococcus pneumoniae, Guillain-Barré syndrome, M. bovis, salmonella, and many other polysaccharide or glycolipid components of bacterial and viral infectious agents.

In the foregoing details, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as critical, required, or essential features or elements of any or all the claims. The invention is defined solely by the appended claims including any amendments made during the pendency of this application and all equivalents of those claims as issued.

What is claimed is:

1. A method for pretreatment of proteins present in an undiluted body fluid sample from humans and animals, the method comprising the steps of:
providing an enzyme-modified solid support with immobilized enzymes;
flowing the body fluid sample across the enzyme-modified solid support;
digesting the proteins in the body fluid sample by the peptide cleavage action of the immobilized enzymes; and
heating the body fluid sample post-digestion to remove peptide fragments that can interfere with downstream analysis.

2. The method of claim 1, wherein the solid support is inert to the immobilized enzymes and the body fluid sample.

3. The method of claim 1, wherein the solid support is a membrane, a fiber, a mesh, a capillary, particles, or beads.

4. The method of claim 1, wherein the immobilized enzymes comprise serine proteases including but not limited to proteinase K.

5. The method of claim 1, further comprising a step of controlling a temperature of the immobilized enzymes and the body fluid sample.

6. The method of claim 1, further comprising a step of controlling a loading of the immobilized enzymes.

7. The method of claim 1, further comprising a step of controlling an incubation time of the body fluid sample over the immobilized enzymes.

8. The method of claim 1, wherein the body fluid sample comprises serum, plasma, whole blood, urine, cerebrospinal fluid, saliva, interstitial fluid, or nasal fluids.
